SEQUENCE LISTING (9 sequences)

SEQ ID NO: 1 - DNA, 16 bp, Unknown
  PCR Primer
cgggccctggcaaaac

SEQ ID NO: 2 - DNA, 16 bp, Unknown
  PCR Primer
cgccctcgcctttgct

SEQ ID NO: 3 - DNA, 34 bp, Unknown
  PCR Primer
tttgtatgactcatacataaagtccttcccgctg

SEQ ID NO: 4 - DNA, 20 bp, Unknown
  PCR Primer
cccagaaagcgattccttca

SEQ ID NO: 5 - DNA, 22 bp, Unknown
  PCR Primer
aatggcatctctgtgtcaacca

SEQ ID NO: 6 - DNA, 37 bp, Unknown
  PCR Primer
tgatacactgtctgcaaacatatcacaagaaatgacc

SEQ ID NO: 7 - DNA, 17 bp, Unknown
  PCR Primer
gcgcggctacagcttca

SEQ ID NO: 8 - DNA, 22 bp, Unknown
  PCR Primer
tctccttaatgtcacgcacgat

SEQ ID NO: 9 - DNA, 18 bp, Unknown
  PCR Primer
caccacggccgagcggga

(19) United States
(12) Patent Application Publication
(10) Pub. No.: US 2007/0104714 A1
(43) Pub. Date: May 10, 2007

(54) Composition and method for the treatment of cancer and other physiologic conditins based on modulation of the ppar-gamma pathway and her-kinase axis

(75) Inventors: David B. Agus, Beverly Hills, CA (US); Anjali Jain, Los Angeles, CA (US); Michael Hedvat, Encino, CA (US)

Correspondence Address: DAVIS WRIGHT TREMAINE LLP, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 10/568,669

(22) PCT Filed: Aug. 27, 2004
(86) PCT No.: PCT/US04/28071

§ 371 Date: Nov. 8, 2006

Related U.S. Application Data
(60) Provisional application No. 60/498,849, filed on Aug. 29, 2003.
(60) Provisional application No. 60/568,910, filed on May 7, 2004.

(51) Int. Cl. A61K 39/395; A61K 31/60; A61K 31/405; A61K 31/192

(52) U.S. Cl. 424/146.1; 514/165; 514/420; 514/569; 514/570

(57) ABSTRACT

Described herein are methods of using a NSAID and a HER-kinase axis inhibitor in the treatment of various conditions including cancer, and especially prostate, breast, lung, ovarian, brain and colon cancers, through regulation of PPARγ activity. In various embodiments, the NSAID and HER-kinase axis inhibitor may be included in a composition that is useful for the treatment of conditions in a mammal. Also described is a kit including a NSAID and a HER-kinase axis inhibitor along with instructions for their use in treating and preventing disease conditions, such as cancer.

### FIELD OF INVENTION

Embodiments of the present invention are directed to methods for treating and preventing disease conditions that are modulated by the PPARY pathway and HER-kinase axis, such as cancer.

### BACKGROUND OF THE INVENTION

Cancer is the second leading cause of death in the United States, and over one million people are diagnosed with cancer each year. Approximately one out of every two American men and one out of every three American women will have some type of cancer during their lifetime. However, while substantial progress has been made in identifying some of the likely environmental and hereditary causes of cancer, the morbidity rates associated with this disease indicate a need for substantial improvement in the therapeutic interventions for cancer and related diseases and disorders.

Peroxisome proliferator activated receptors (PPARs) are members of the nuclear hormone receptor superfamily and act as ligand-induced transcription factors. Upon ligand stimulation, these receptors form heterodimers with a retinoid X receptor (RXR), and bind to peroxisome proliferator response elements (PPREs), modulating the rate of transcription of target genes (Sporn, M. B. et al, “Prospects for Prevention and Treatment of Cancer with Selective PPARγ Modulators (SPARMs),” Trends Mol. Med., Vol. 7, No. 9, p. 395-400 (2001)). A well documented mechanism utilized to negatively regulate PPAR transcriptional activity is via phosphorylation of PPAR by mitogen activated protein (MAP) kinase (Camp, H. S. and Tafuri, S. R., “Regulation of Peroxisome Proliferator-Activated Receptor γ Activity by Mitogen-Activated Protein Kinase,” J. Biol. Chem., Vol. 272, No.16, p. 10811-10816 (1997); Floyd, Z. E. and Stephens, J. M., “Interferon-γ-Mediated Activation and Ubiquitin-Proteasome-Dependent Degradation of PPARγ in Adipocytes,” J. Biol. Chem., Vol. 277, No. 6, p. 4062-4068 (2002); Hauser, S. et al, “Degradation of the Peroxisome Proliferator-Activated Receptor γ is Linked to Ligand-Dependent Activation,” J. Biol. Chem., Vol. 275, No. 24, p. 18527-18533 (2000)). This phosphorylation event reduces ligand binding affinity of PPAR, and leads to a downregulation of the PPAR protein levels by ubiquitin-proteosome-mediated-degradation (Floyd, Z. E. and Stephens, J. M.; Shao, D. et al., “Interdomain Communication Regulating Ligand Binding by PPARγ,” Nature, Vol. 396, p. 377-380 (1998)).

The transcription factor PPARγ has been described as an important anti-tumorigenic molecule involved in the control of cell growth and differentiation (Tontonoz, P. et al., “mPPARγ2: Tissue-Specific Regulator of an Adipocyte Enhancer,” Genes & Dev., Vol. 8, p. 1225-1234 (1994)). PPARγ has been divided into three sub-types, PPARγ1, PPARγ2, and PPARγ3 that are derived from distinct transcription start sites followed by alternative splicing events (Fajas, L. et al., “PPARγ3 mRNA: A distinct PPARγ mRNA Subtype Transcribed from an Independent Promoter,” FEBS Letters, Vol. 438, p. 55-60 (1998); Green, S., “PPAR: A Mediator of Peroxisome Proliferator Action,” Mutat. Res., Vol. 333, Nos. 1, 2, p. 101-109 (1995); Zhu, Y. et al., “Structural Organizations of Mouse Peroxisome Proliferator-Activated Receptor γ (mPPARγ) Gene: Alternative Promoter Use and Different Splicing Yield Two mPPARγ Isoforms,” Proc. Natl. Acad. Sci. USA, Vol. 92, p. 7921-7925 (1995)). No significant functional difference between the PPARγ variants has been reported, however the relative expression of these variants is tissue specific. For example, PPARγ1 appears to be expressed in several tissues, whereas PPARγ2 is expressed primarily in adipose tissue (Fajas, L. et al., “The Orgainization, Promoter Analysis, and Expression of the Human PPARγ Gene,” J. Biol. Chem., Vol. 272, No. 30, p. 18779-18789 (1997)). Studies have shown that activation of PPARγ in certain tissues blocks the cell cycle, retards growth, and induces cell differentiation (Tontonoz, P. et al., “Stimulation of adipogenesis in fibroblasts by PPAR gamma 2, a lipid-activated transcription factor,” Cell, Vol. 79, p. 1147-1156 (1994);Tontonoz, P. et al., “Terminal Differentiation of Human Liposarcoma Cells Induced by Ligands for Peroxisome Proliferator-Activated Receptor γ and the Retinoid X Receptor,” Proc. Natl. Acad. Sci. USA, Vol. 94, p. 237-241 (1997); Elstner, E. et al., “Ligands for peroxisome proliferator-activated receptor gamma and retinoic acid receptor inhibit growth and induce apoptosis of human breast cancer cells in vftro and in BNX mice,” Proc. Natl. Acad. Sci. USA, Vol. 95, p. 8806-8811 (1998); Kubota, T. et al., “Ligand for peroxisome proliferator-activated receptor gamma (troglitazone) has potent antitumor effect against human prostate cancer both in vitro and in vivo,” Cancer Res., Vol. 58, p. 3344-3352 (1998); Sarraf, P. et al., “Differentiation and reversal of malignant changes in colon cancer through PPARgamma,” Nat. Med., Vol. 4, p. 1046-1052 (1998)). Such observations have encouraged research into the development of novel PPARγ ligands as chemopreventative agents in the treatment of carcinogenesis.

Studies of prostate cancer have revealed that PPARγ is significantly expressed in primary prostate cancer but at very low levels in normal prostate tissue, making it a promising candidate for molecular therapy (Segawa, Y. et al., “Expression of Peroxisome Proliferator-Activated Receptor (PPAR) in Human Prostate Cancer,” Prostate, Vol. 51, p. 108-116 (2002)). Furthermore, hemizygous deletion of PPARγ is common in primary human prostate cancers, suggesting that loss of function may contribute to the malignant phenotype (Mueller, E. et al., “Effects of Ligand Activation of Peroxisome Proliferator-Activated Receptor γ in Human Prostate Cancer,” Proc. Natl. Acad. Sci. USA, Vol. 97, No. 20, p. 10990-10995 (2000)). In accordance with this observation, it was found that PPARγ activation with synthetic ligands downregulates prostate specific antigen (PSA) mRNA expression in prostate cancer cells in vitro (Hisatake, J. I. et al., “Down-Regulation of Prostate-Specific Antigen Expression by Ligands for Peroxisome Proliferator-Activated Receptor γ Human Prostate Cancer,” Cancer Res., Vol. 60, p. 5494-5498 (2000); Kubota, T. et al., “Ligand for Peroxisome Proliferator-Activated Receptor γ (Troglitazone) has Potent Antitumor Effect Against Human Prostate Cancer Both In vitro and In vivo,” Cancer Res., Vol. 58, p. 3344-3352 (1998)). Specifically, PPARγ ligands, troglitazone, rosiglitazone and 15-deoxy-12, 14-prostaglandin J2 have demonstrated growth inhibition of prostate cancer cell lines that express an appreciable level of PPARγ (Mueller et al., 2000; Segawa et al., “Expression of peroxisome proliferator-activated receptor (PPAR) in human prostate cancer,” Prostate, Vol. 51, p. 108-116 (2002)). Moreover, a phase 11 clinical study of troglitazone treatment in patients with prostate cancer was associated with prolonged periods of stable disease characterized by the absence of new metastases or disease-related symptoms and lower PSA levels (Mueller et al., 2000). Troglitazone (available under the trade name REZULIN from Parke-Davis division of Wamer-Lambert Company; Morris Plains, N.J.) is a synthetic ligand that downregulates PSA mRNA expression.

One therapeutic treatment known to act on the PPARγ is the group of non-steroidal anti-inflammatory drugs, otherwise known as “NSAIDS”. Recently, attention has been focused on the anti-proliferative activity of NSAIDS in cancerous or transformed cells, which is mediated through an interaction with PPARγ (Na, H. K. and Y. J. Surh, “Peroxisome proliferator-activated receptor gamma (PPARgamma) ligands as bifunctional regulators of cell proliferation,” Biochem. Pharmacol., Vol. 66, p. 1381-1391 (2003)). Etodolac is one such NSAID (Demerson et al., “Resolution of etodolac and antiinflammatory and prostaglandin synthetase inhibiting properties of the enantiomers,” J. Med. Chem., Vol. 26, p. 1778-1780 (1983)). Etodolac is pyranocarboxylic acid, chemically designated as (±) 1,8-diethyl-1,3,4,9-tetrahydropyrano-[3,4-b]indole-1-acetic acid. It exhibits anti-inflammatory, analgesic, and antipyretic activities. The molecular basis for the therapeutic actions of NSAIDs are believed to be through inhibition of cyclooxygenase (COX) activity, thereby blocking the production of prostaglandins (PGs). However, there is a need to determine the chemopreventative and anti-metastatic effects of NSAIDs that can be separated from COX inhibition because COX inhibition leads to many undesirable side-effects (Allison et al., 1992). The etodolac analog, R-etodolac, lacks COX inhibitory activity and is considered the “inactive” enantiomer of this drug (Adachi et al., “Apoptosis induced by molecular targeting therapy in hematological malignancies,” Acta. Haematol., Vol. 111, p. 107-123 (2004); Demerson et al., “Resolution of etodolac and antiinflammatory and prostaglandin synthetase inhibiting properties of the enantiomers,” J. Med. Chem., Vol. 26, p. 1778-1780 (1983)). It was recently demonstrated to inhibit transcription of a β-catenin-dependent T cell and lymphoid enhancing transcription factor (TCF/LEF) reporter gene in HEK293 cells, and at the same concentrations, diminished the in vitro survival of chronic lymphocytic leukemia (CLL) cells (Lu et al., “Activation of the Wnt signaling pathway in chronic lymphocytic leukemia,” Proc. Natl. Acad. Sci. USA, Vol. 101, p. 3118-3123 (2004)). R-etodolac is currently being tested in Phase II clinical trials for treating CLL.

Another promising set of targets for therapeutic intervention in the treatment of cancer includes the members of the human epidermal growth factor receptor (HER) or ErbB family of receptor tyrosine kinases (RTKs), which are important mediators of cell growth, survival, and differentiation. They are frequently upregulated in solid epithelial tumors of, by way of example, the prostate, lung and breast, and are also upregulated in glioblastoma tumors. The HER-kinase signaling network is active in prostate cancer cells and is known to contribute significantly to the progression of the disease (Agus, D. B. et al., “Targeting Ligand-Activated ErbB2 Signaling Inhibits Breast and Prostate Tumor Growth,” Cancer Cell, Vol. 2, p. 127-137 (2002)). The signaling network is activated by receptor-specific ligand stimulation that leads to receptor dimerization and autophosphorylation. Therapies directed against the HER-kinase axis, such as recombinant humanized monoclonal antibody 2C4, or rhuMab 2C4 (hereinafter, “2C4”), inhibit the growth of prostate xenografts by as much as 80%, corroborating the concept that the HER-kinase axis is an important mediator of prostate cancer growth (Agus et al., 2002). In vivo studies have demonstrated that ligands for the HER-kinase axis induce phosphorylation of PPARγ by mitogen-activated protein (MAP) kinase, which may eventually regulate the levels of PPARγ (Camp, H. S. and Tafuri, S. R., “Regulation of peroxisome proliferator-activated receptor gamma activity by mitogen-activated protein kinase,” J. Biol Chem., Vol. 272, p. 10811-10816 (1997)). However, none of the studies have suggested a direct modulation of the HER-kinase axis by a PPARγ ligand.

A significant limitation in therapeutic treatments directed exclusively at either the PPARγ pathway or the HER-kinase axis is that recipients thereof tend to develop a resistance to their therapeutic effects after they initially respond to therapy. Although these treatments may, at first, exhibit strong anti-tumor properties, they may soon become less potent or entirely ineffective in the treatment of cancer. In addition, the biomolecular and pathological mechanism responsible for this resistance has not been elucidated in the past by medical research, leaving patients who have exhibited such resistance with few alternative therapeutic treatments. Without an understanding of the mechanisms of both the PPARγ pathway and the HER-kinase axis, the therapeutic and diagnostic potential of such treatments is largely untapped.

There is a significant need in the art for a satisfactory treatment for cancer, which incorporates the benefits of activation of both the HER-kinase axis and PPARγ pathways, and that overcomes the resistance exhibited by many patients.

### SUMMARY OF THE INVENTION

Described herein is a composition useful for treating conditions in a mammal. This composition includes a non-steroidal anti-inflammatory drug (NSAID) and a HER-kinase axis inhibitor, which may be administered to a mammal by any conventional means, such as, by way of example, oral gavage or intraperitoneal injection. The composition of the present invention may further include an additional component such as an adjuvant, to provide a therapeutically convenient formulation and/or to enhance biochemical delivery and efficacy of the composition. Methods of treating or preventing cancer with the NSAID and HER-kinase axis inhibitor of the present invention are also provided.

Embodiments of the present invention additionally provide methods for modulating a PPARγ pathway in a mammal and for treating conditions in a mammal. The methods of the present invention include the combined use of a NSAID and a HER-kinase axis inhibitor, which can be administered by any conventional means, such as, by way of example, oral gavage or intraperitoneal injection. Further, the NSAID and HER-kinase axis inhibitor can be administered at different time intervals or separately from one another, and may be delivered by different means. The NSAID and HER-kinase axis inhibitor may each further include an additional component such as an adjuvant, to provide a therapeutically convenient formulation and/or to enhance biochemical delivery and efficacy of the composition. The methods of the present invention may be useful in the treatment of disease conditions, such as cancer.

Further embodiments of the present invention provide a kit for use in a mammal comprising a NSAID and HER-kinase axis inhibitor. The kit of the present invention includes the use of a NSAID and HER-kinase axis inhibitor in a manner consistent with the methods of the present invention.

### BRIEF DESCRIPTION OF THE DRAWINGS

FIG. 1 demonstrates the ability of R-etodolac to transactivate PPARγ, in accordance with an embodiment of the present invention. FIG. 1A is a graphical representation of R-etodolac transactivation of a PPARγ reporter construct (AOx)3-TK-Luc by increasing concentrations of etodolac, indomethacin, and rosiglitazone in RAW 267.4 mouse macrophages. The results are presented as a fold-induction of luciferase expression relative to the no drug treatment control and are expressed as the mean±one standard of deviation (SD) of three separate experiments. Transfection efficiency was normalized using a CMV-β-gal reporter construct. FIG. 1B is a graphical representation of the enhancement of phorbol ester (TPA) induced expression of the CD36 scavenger receptor in THP-1 cells by etodolac and troglitazone (TGZ). An increase in CD36 expression was determined by flow cytometry in the presence or absence of TPA with the indicated drugs. The results are presented as the difference in mean fluorescence between anti-CD36 antibody and control isotype-matched antibody. FIG. 1C displays photographs of lipid accumulation in NIH3T3-PPARγ cells induced by R-etodolac. NIH3T3 cells expressing recombinant PPARγ were treated for seven days with (i) vehicle (DMSO) alone, (ii) 1 μM Troglitazone, (iii) 1 μM R-etodolac or (iv) 500 μM R-etodolac. The cells were stained for neutral lipids with Oil Red O stain. The photographs are displayed as a 400X magnification. The dark spots indicate the accumulation of neutral lipids.

FIG. 2 demonstrates the effect of R-etodolac on PPARγ1 positive prostate cancer xenografts, in accordance with an embodiment of the present invention. FIG. 2A depicts a representative western blot analysis showing PPARγ1 expression in protein lysates prepared from androgen independent CWRSA6 and androgen dependent LuCaP-35 human prostate cancer xenografts. FIG. 2B is a graphical representation of the response of CWRSA6, or LuCaP-35 tumors to R-etodolac (●) administered at a dosage of 200 mg/kg via daily o.g. or vehicle treated control (∘). Results are presented as mean tumor volume (n=10)±SE.

FIG. 3 evidences the reduction in Cyclin D1 mRNA and protein expression in LNCaP cells treated with R-etodolac, in accordance with an embodiment of the present invention. FIG. 3A is a graphical representation of a real-time quantitative RT-PCR assay demonstrating cyclin D1 mRNA expression in LNCaP cells treated with increasing concentrations of R-etodolac (0, 200 and 400 μM) for 18 hours. The levels of RNA were normalized using an assay for 18S RNA. The level of cyclin D1 transcripts in untreated cells was set to 100 percent. This graph represents the mean from three independent experiments ±SD. FIG. 3B depicts a Western blot analysis demonstrating cyclin D1 protein expression in LNCaP cells treated with increasing concentrations of R-etodolac (0, 200, 400, and 600 μM) for 18 hours. As a normalization control, β-actin protein was used. The figure is representative of at least 3 independent assays.

FIG. 4 evidences the degradation of PPARγ protein following R-etodolac treatment in CWRSA6 and LuCaP-35 prostate cancer xenografts, in accordance with an embodiment of the present invention. FIG. 4A depicts a Western blot analysis demonstrating PPARγ1 expression in protein lysates prepared from the R-etodolac efficacy studies involving the CWRSA6 and LuCaP-35 human prostate cancer xenografts. Lanes 3, 4, 7, and 8 represent vehicle treated control tumors, whereas lanes 1 and 2 represent CWRSA6 tumors following 19 days of treatment with R-etodolac, and lanes 5 and 6 represent LuCaP-35 tumors following 13 days of treatment with R-etodolac. Equal protein loading was confirmed with a β-actin antibody. FIG. 4B displays a real-time quantitative RT-PCR analysis of PPARγ1 mRNA expression in CWRSA6 and LuCaP-35 xenografts following the animal efficacy studies. Levels of PPARγ mRNA expression were normalized to β-actin mRNA levels, and results are displayed as an average (n=5) relative percentage to control animals ± SD. No significant difference was observed between treated and non-treated control animals.

FIG. 5 demonstrates the effect of R-etodolac treatment in increasing phospho-ERK activity in CWRSA6 xenografts and 22Rv1 prostate cancer cells, in accordance with an embodiment of the present invention. FIG. 5A depicts a Western blot analysis of lysates prepared from R-etodolac treated CWRSA6 xenografts following the time course experiment as described in the material and methods. Animals received R-etodolac every 24 hours; lanes 1 and 2 represent tumors following 24 hours of treatment, lanes 4 and 5 represent tumors following 48 hours of treatment, and lanes 5 and 6 represent tumors following 72 hours of treatment with R-etodolac. Lanes 7 and 8 represents vehicle treated control animals following 72 hours of treatment. Equal loading was confirmed with a β-actin antibody, as well as with an antibody against total MAP kinase. FIG. 5B depicts whole cell lysates prepared from 22Rv1 cells treated with R-etodolac (500 μM) for various times shown. The representative experiment was repeated independently three times.

FIG. 6 demonstrates that inhibition of MAP kinase by 2C4 prevents R-etodolac induced degradation of PPARγ protein thus promoting efficacy of R-etodolac, in accordance with an embodiment of the present invention. FIG. 6A is a graphical representation of the response of CWRSA6 tumors to R-etodolac (●) administered at 200 mg/kg via daily o.g., or 2C4 (▪) administered at 20 mg/kg via inter-peritoneal injection, or a combination regimen of R-etodolac and 2C4 (□) at the previously stated doses. The arrow indicates the initiation of therapy. Results are presented as mean tumor volume (n=10)± SD. FIG. 6B depicts a Western blot analysis showing PPARγ1 expression in lysates prepared from the R-etodolac and 2C4 combination efficacy studies involving the androgen independent CWRSA6 human prostate xenograft tumors following 22 days of treatment. Lanes 1 and 2 represent R-etodolac treated tumors, lanes 3 and 4 represent tumors receiving 2C4 alone, and lanes 5 and 6 represent tumors receiving a combination of R-etodolac and 2C4.

### DETAILED DESCRIPTION OF THE INVENTION

The present invention is based on the surprising discovery that R-etodolac, a stable stereoisomer of a non-steroidal anti-inflammatory drug (NSAID) and a novel PPARγ modulator, can effectively suppress the growth of prostate cancer xenografts without apparent morbidity by modulating both the PPARγ pathway and the HER-kinase axis. (Hedvat, M. et al., “Inhibition of HER-kinase activation prevents ERK-mediated degradation of PPARγ,” Cancer Cell, Vol. 5, No. 6, p. 565-574 (2004)). The clinical utility of this cross-talk is demonstrated by additive growth inhibitory properties of R-etodolac and 2C4, a HER-2 specific monoclonal antibody currently in Phase II clinical trials. “Conditions” and “disease conditions,” as used herein may include, but are in no way limited to cancer, and especially prostate, breast, lung, ovarian, brain and colon cancers. “Treatment” and “treating,” as used herein include preventing, inhibiting, curing, and alleviating cancer or other disease conditions or symptoms thereof, and preventing, inhibiting, curing and alleviating the metastasis of cancer. “Beneficial results” may include, but are in no way limited to, lessening the severity of the disease condition, preventing the disease condition from worsening, curing the disease condition and prolonging a patient's life or life expectancy. The disease conditions relate to or are modulated by the PPARγ pathway, the HER-kinase axis or a combination thereof. “Alleviating” specific cancers includes degrading a tumor, for example, breaking down the structural integrity or connective tissue of a tumor, such that the tumor size is reduced when compared to the tumor size before treatment. “Alleviating” metastasis of cancer includes reducing the rate at which the cancer spreads to other organs. “Preventing” metastasis of cancer includes preventing the cancer from spreading outside of a specific tissue. “Curing” cancer includes degrading a tumor such that a tumor cannot be detected after treatment. The tumor may be reduced in size or become undetectable, for example, by atrophying from lack of blood supply or by being attacked or degraded by one or more components administered according to the invention.

Successful use of R-etodolac in a combination treatment regimen with 2C4 demonstrated the potential utility of 2C4 to increase sensitivity of epithelial cancers to PPARγ ligands. While not wishing to be bound by any theory, it is believed that the primary mechanism of anti-tumor activity exerted by R-etodolac is through the enhancement of transcriptional activity of the PPARγ receptor. PPARγ is well documented to have an effect on cell cycle progression through the repression of the cyclin D1 promoter (Wang et al., 2001). Cyclin D1 determines the rate of progression of mammary epithelial cells through the G1 phase in response to mitogenic and oncogenic signals (Lee et al., 2000). Furthermore, it is believed that R-etodolac inhibits the progression of prostate cancer by downregulating cyclin D1 expression via the PPARγ pathway. R-etodolac may also contribute to tumor inhibition via upregulation of apoptosis via the Wnt/β-catenin pathway (Lu et al., 2004).

However, PPARγ protein degradation is observed post-R-etodolac treatment. This loss of PPARγ protein is a post-transcriptional event because no change in PPARγ mRNA expression is observed following transcription. Furthermore, the lack of PPARγ protein is not due to selection against PPARγ positive cells. Selection against PPARγ positive cells was ruled out based on the results of the combination drug therapy study discussed infra. The other conclusion, that the anti-tumorigenic effect of R-etodolac works through loss of PPARγ protein, was refuted by immunoblots of the time course experiment, revealing an upregulation of the PPARγ receptor in the majority of the animals 24 to 72 hours post R-etodolac treatment. The time period of PPARγ upregulation in xenografts receiving R-etodolac coincides with the peak in PPARγ transcriptional activity in cell culture based assays following R-etodolac administration. These results strengthen the idea that the initial effect of R-etodolac is to increase PPARγ activity. The belief is that the observed loss of PPARγ protein is subsequent to PPARγ activation since an essential requirement for PPARγ degradation is the ligand induced conformational change of the receptor, which is associated with transcriptional activation (Hauser et al., 2000).

A well documented mechanism utilized to negatively regulate PPARγ transcriptional activity is via phosphorylation of PPARγ by MAP kinase (Camp and Tafuri, 1997; Floyd and Stephens, 2002; Hauser et al., 2000). This phosphorylation event reduces ligand binding affinity of PPARγ, and leads to a downregulation of the PPARγ protein levels by ubiquitin-proteosome-mediated-degradation (Floyd and Stephens, 2002; Shao et al., 1998). Phosphorylation has also been documented for regulation of the progesterone receptor (Lange et al., 2000; Shen et al., 2001). The observation that treatment with R-etodolac leads to an upregulation of phospho-MAP kinase and a subsequent loss of PPARγ protein suggests a similar mechanism of regulation. It is hypothesized that the loss of PPARγ protein, a target of R-etodolac, may be a mechanism of “auto-resistance” to the drug. The loss of PPARγ protein would also compromise the ability of R-etodolac to repress cyclin D1. Additionally, induction of phospho-MAP kinase by R-etodolac would lead to an increase in cyclin D1 since MAP kinase induces the cyclin D1 promoter through a phospho-PPARγ independent mechanism (Wang et al., 2001; Watanabe et al., 1998). Increased cyclin D1 is documented to repress PPARγ expression and transactivation, limiting its anti-tumorigenic potential (Wang et al., 2003). These events would add to the mechanism of auto-resistance of R-etodolac.

It has been discovered that the auto-resistance phenomenon can be overcome by use of a combination regimen of R-etodolac and 2C4. The combination regimen promotes maintenance of PPARγ protein in the xenograft, which correlates with increased anti-tumorigenicity. It is believed that treatment with 2C4 in combination with R-etodolac also perpetuates the inhibition of cyclin D1 through the maintenance of the PPARγ protein and through the abrogation of phospho-MAP kinase activity, thus overcoming the auto-resistance to R-etodolac. The combination study, disclosed herein, also supports the notion that the absence of PPARγ expression in R-etodolac treated xenografts is not the result of selection against PPARγ positive cells. Taken together, these observations suggest that R-etodolac-mediated growth inhibition is achieved through activation of the PPARγ receptor while controlling this activity through a mechanism of auto-resistance. This combination therapy may be particularly effective in the treatment of cancer and other conditions that may benefit from modulation of these axes.

The results of the combination therapy suggest that R-etodolac activates phospho-MAP kinase through stimulation of the HER-kinase axis. The HER-kinase axis is a positive regulator of prostate cancer cell survival as demonstrated by growth inhibition curves of prostate xenografts (80%) by the HER-2 specific monoclonal antibody, 2C4 (Agus et al., 2002). The studies disclosed herein show 2C4 rescues PPARγ protein from degradation and that 2C4 growth inhibition is additive to that achieved with R-etodolac alone. In addition to lending support for the proposed model of auto-resistance of R-etodolac, the studies suggest the involvement of the HER-kinase axis in PPARγ degradation. Other studies have shown that HER-2 mRNA overexpression enhances the expression of PPARγ (Yang et al., 2003), however, the current studies did not show change in HER-2 receptor levels. While R-etodolac treatment did not alter the mRNA levels of HER-kinase receptors (data not shown), it is possible that the PPARγ transactivator renders the receptors constitutively active or increases the concentration of endogenous ligands. For example, in an analogous system, activation of MAP kinase by ligands such as epidermal growth factor and platelet derived growth factor induce the phosphorylation of PPARγ1 on Ser82 and decrease its ability to activate transcription (Camp and Tafuri, 1997). Thus, these observations reveal an important crosstalk between the HER-kinase axis and PPARγ pathway in prostate tumorigenesis.

The present invention, in one embodiment, relates to a composition useful in affecting a PPARγ pathway-sensitive condition, wherein the composition includes a NSAID and a HER-kinase axis inhibitor. Furthermore, the NSAID and HER-kinase axis inhibitor of the composition may be suitable for use as a single agent, any suitable formulation with one another, or with additional NSAIDs or HER-kinase axis inhibitors as would be readily recognized by one of skill in the art.

The NSAIDs used in connection with various embodiments of the present invention may exhibit anti-cancer properties. The NSAID may be R-etodolac or a R-etodolac derivative, but may also include, without limitation, aspirin, diclofenac, diflunisal, etodolac, fenoprofen, floctafenine, flurbiprofen, ibuprofen, indomethacin, ketoprofen, meclofenamate, mefenamic acid, meloxicam, nabumetone, naproxen, oxaprozin, piroxicam, sunlindac, tenoxicam, tiaprofenic acid, tolmetin, and pharmaceutical equivalents, derivatives and salts, as well as other functionally related compounds, although numerous other NSAIDs may be used, as will be readily appreciated by those of skill in the art. For example, guidance as to particular NSAIDS is provided in the literature and generally available to practitioners in the art. See, e.g., U.S. Pat. No. 6,761,913 (describing the use of celery seed extracts and additional NSAIDS for the treatment of inflammation, and U.S. Pat. No. 6,759,056 (describing a transdermal delivery system incorporating numerous NSAIDs). As further described in the ensuing examples, R-etodolac was found to upregulate PPARγ and inhibit tumor growth prostate cancer xenografts, and it was generally well-tolerated by recipients.

The HER-kinase axis inhibitors used in connection with various embodiments of the present invention may exhibit anti-cancer properties. The HER-kinase axis inhibitor may be 2C4 or a 2C4 derivative, but may also include, without limitation, ansamycins, gefitinib (compound ZD1839 developed by AstraZeneca UK Ltd.; available under the tradename IRESSA; hereinafter “IRESSA”), erlotinib (compound OSI-774 developed by Genentech, Inc. and OSI Pharmaceuticals, Inc.; available under the tradename TARCEVA; hereinafter “TARCEVA”), monoclonal antibodies, rapamycin, src (transforming gene of Rous sarcoma virus) inhibitors, tyrosine kinase inhibitors, LY294002 (available from Cayman Chemical), imatinib mesylate (available from Novartis Pharmaceuticals Corp. under the tradename GLEEVEC; hereinafter “GLEEVEC”), trastuzumab (available from Genentech, Inc. under the tradename HERCEPTIN; hereinafter “HERCEPTIN”), CI1033 (available from Pfizer Inc.), PKI166 (available from Novartis AG), GW2016 (available from GlaxoSmithKline), EKB569 (available from Wyeth), IMC-C225 (available from ImClone Systems Inc. and Bristol-Myers Squibb Co.), and pharmaceutical equivalents, derivatives and salts, as well as other functionally related compounds, although numerous other HER-kinase axis inhibitors may be used, as will be readily appreciated by those of skill in the art. For example, guidance as to particular HER-kinase axis inhibitors is provided in the literature and generally available to practitioners in the art. See, e.g., U.S. patent application No. 2002/0045570 (describing compositions for the inhibition of HER-family tyrosine kinases). As further described in the ensuing examples, 2C4 was found to inhibit MAP-kinase phosphorylation of PPARγ, thereby preventing PPARγ protein degradation, and it was generally well tolerated by recipients.

In another aspect of the present invention, a method for treating cancer or cancerous tumors in mammals is provided. The method may include providing an NSAID; providing a HER-kinase axis inhibitor; and implementing a combination therapy to the recipient in a manner to treat the particular condition. Furthermore, the NSAID and HER-kinase axis inhibitor may have characteristics similar to the compositions described above in accordance with alternate embodiments of the present invention.

Notably, the methods of the present invention are not limited to the treatment of cancer. Instead, it will be readily understood that the biomolecular pathways addressed and the cross-talk between the PPARγ pathway and HER-kinase axis obviated by the methods of the present invention may find application in the treatment of other disease conditions; any disease condition in which treatment with a combination therapy of a NSAID and HER-kinase axis inhibitor may cause a beneficial result for a patient is thus included within the scope of the present invention.

There are various reasons why one might wish to administer a composition including both a NSAID and a HER-kinase axis inhibitor of the present invention rather than administering these compounds separately in a combination therapy. Depending on the particular NSAID or HER-kinase axis inhibitor that one uses, a composition might have superior characteristics as far as clinical efficacy, solubility, absorption, stability, toxicity and/or patient acceptability are concerned. It will be readily apparent to one of ordinary skill in the art how one can formulate a composition of any of a number of combinations of NSAIDs and HER-kinase axis inhibitors of the present invention. There are many strategies for doing so, any one of which may be implemented by routine experimentation. However, the pharmacokinetics of the NSAID and HER-kinase axis inhibitor of the invention may be more suitable for separate administration of the compounds.

In any of the embodiments of the invention, the compositions of the invention can be administered in combination with other appropriate therapeutic treatments. For example, the NSAID and HER-kinase axis inhibitor of the present invention may be administered in addition to an established therapy, such as chemotherapy, radiation treatment or any other therapy known in the art to treat cancer or another PPARγ pathway-sensitive condition. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects.

The determination of a therapeutically effective dose is well within the capability of those skilled in the art. A “therapeutically effective” dose refers to that amount of active ingredient which increases or decreases the effects of a disease condition relative to that which occurs in the absence of the therapeutically effective dose. Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀. Furthermore, the data obtained from cell culture assays and animal studies is used in formulating a range of dosage for human use, which can be readily tended to by one of ordinary skill in the art without undue experimentation. The dosage contained in such compositions may be selected so as to be within a range of circulating concentrations that include the ED₅₀ with little or no toxicity. The dosage varies within this range depending upon the dosage form employed, sensitivity of the patient, and the route of administration.

The appropriate dosage of the NSAID and HER-kinase axis inhibitor of the invention may depend on a variety of factors. Such factors may include, but are in no way limited to, a patient's physical characteristics (e.g., age, weight, sex), whether the compound is being used as single agent or adjuvant therapy, the type of PPARγ pathway-sensitive condition being treated, the progression (i.e., pathological state) of the cancer or other PPARγ pathway-sensitive condition, and other factors that may be recognized by one skilled in the art. Furthermore, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans. However, the exact dosage will be determined by the practitioner, in light of factors related to the subject that requires treatment.

The NSAID is generally administered via oral gavage at an amount of at least about 200 mg/kg; for instance, about 240 mg/kg. Suitable amounts ordinarily range from about 100 mg/kg to about 500 mg/kg. The HER-kinase axis inhibitor is generally administered via intraperitoneal injection at an amount of at least about 18 mg/kg; for instance, about 20 mg/kg. Suitable amounts ordinarily range from about 5 mg/kg to about 40 mg/kg. Other ranges within the ranges expressly disclosed above may also be suitable. For example, guidance as to particular dosages and methods of delivery is provided in the literature and generally available to practitioners in the art.

The administration of the NSAID and HER-kinase axis inhibitor of the invention may include, without limitation, delivery of the compounds together, delivery of each compound separately, delivery as a single dosage, delivery periodically, or delivery of the compounds separately and/or at different intervals, although other schemes of administration may be used, as will be readily appreciated by those skilled in the art. “Periodically,” as used herein includes, but is in no way limited to, any interval of time such as hourly, daily, weekly, twice weekly, and monthly as would be recognized by one skilled in the art. In those embodiments wherein the compounds of the present invention are formulated for administration separately and at different intervals, the NSAID is administered on a NSAID periodic basis and the HER-kinase axis inhibitor is administered on a HER-kinase axis inhibitor periodic basis. “NSAID periodic basis,” as used herein includes, but is in no way limited to, any interval of time such as hourly, daily, weekly, twice weekly, and monthly as would be recognized by one skilled in the art. “HER-kinase axis inhibitor periodic basis,” as used herein includes, but is in no way limited to, any interval of time such as hourly, daily, weekly, twice weekly, and monthly as would be recognized by one skilled in the art.

Furthermore, any of the therapeutic methods described above can be applied to any subject in need of such therapy, including, for example, mammals such as dogs, cats, cows, horses, rabbits, monkeys, and humans.

In various embodiments of the invention, the NSAID and HER-kinase axis inhibitor can be formulated as a pharmaceutical composition which can be administered to a patient to achieve a therapeutic effect. Pharmaceutical compositions of the invention can comprise a NSAID and a HER-kinase axis inhibitor, as well as mimetics, agonists, antagonists, or inhibitors of the PPARγ pathway. Any inventive composition described herein can be administered alone or in combination with at least one other agent, such as a stabilizing compound, which can be administered in any sterile, biocompatible pharmaceutical carrier, including, but not limited to, saline, buffered saline, dextrose, and water. The compositions can be administered to a patient alone, or in combination with other agents, drugs or hormones.

In addition to the active ingredients, a pharmaceutical composition can contain suitable pharmaceutically-acceptable carriers including excipients and auxiliaries which facilitate processing of the active compounds into preparations which can be used pharmaceutically. A pharmaceutical composition of the invention can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal delivery routes. A pharmaceutical composition for oral administration can be formulated using pharmaceutically acceptable carriers well known in the art in dosages suitable for oral administration. Such carriers enable a pharmaceutical composition to be formulated as tablets, pills, dragees, capsules, liquids, gels, syrups, slurries, suspensions, and the like, for ingestion by the patient.

A pharmaceutical preparation for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxy-propylmethyl-cellulose, or sodium carboxymethycellulose; gums including arabic and tragacanth; and proteins such as gelatin and collagen. If desired, disintegrating or solubilizing agents can be added, such as the cross-linked polyvinyl pyrrolidone, agar, alginic acid, or a salt thereof, such as sodium alginate.

Dragee cores can be used in conjunction with suitable coatings, such as concentrated sugar solutions, which also can contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound, i.e., dosage.

Pharmaceutical preparations that can be used orally include push-fit capsules made of gelatin, as well as soft, scaled capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

A pharmaceutical formulation suitable for parenteral administration can be formulated in aqueous solutions, preferably in physiologically compatible buffers such as Hanks' solution, Ringer's solution, or physiologically buffered saline. Aqueous injection suspensions can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers also can be used for delivery. Optionally, the suspension also can contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

A pharmaceutical composition of the present invention can be manufactured in a manner that is known in the art, e.g., by conventional mixing, dissolving, granulating, dragee-making, levigating, emulsifying, encapsulating, entrapping, or lyophilizing. A pharmaceutical composition can be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms.

Further details on techniques for formulation and administration can be found in the latest edition of REMINGTON'S PHARMACEUTICAL SCIENCES (Maack Publishing Co., Easton, Pa.). After pharmaceutical compositions have been prepared, they can be placed in an appropriate container and labeled for treatment of an indicated condition. Such labeling may include amount, frequency, and method of administration.

The present invention further provides kits for use within any of the above therapeutic methods. In one embodiment, a kit includes a NSAID, a HER-kinase axis inhibitor and instructions for their use in treating a condition contemplated by the present invention. The exact nature of the components configured in the inventive kit depends on its intended purpose and on the particular methodology that is employed. For example, some embodiments of the kit are configured for the purpose of treating cancer in a subject. In one embodiment, the kit is configured particularly for the purpose of modulating the PPARγ pathway and the HER-kinase axis in a human subject for the treatment of cancer.

Instructions for use may be included with the kit. “Instructions for use” typically include a tangible expression describing the steps for combining a NSAID and a HER-kinase axis inhibitor and/or for using the same in a therapeutic system. Optionally, the kit may also contain other useful components, such as diluents, pharmaceutically acceptable carriers, specimen containers and/or measuring tools.

The materials or components assembled in the kit can be provided stored in any convenient and suitable way that preserves their operability and utility. For example, the components can be in dissolved, dehydrated, or lyophilized form; they can be provided at room, refrigerated, or frozen temperatures.

The components are typically contained in suitable packaging material(s). As employed herein, the phrase “packaging material” refers to one or more physical structures used to house the contents of the kit. The packaging material is constructed by well known methods, preferably to provide a sterile, contaminant-free environment. The packaging materials employed in the kit are those customarily utilized in the field. As used herein, the term “package” refers to a suitable solid matrix or material such as glass, plastic, paper, foil, and the like, capable of holding the individual kit components. Thus, for example, a package can be one or more glass vials used to contain suitable quantities of a NSAID and a HER-kinase axis inhibitor. The packaging material generally has an external label which indicates the contents and/or purpose of the kit and/or its components.

Preferably, the NSAID and HER-kinase axis inhibitor of the invention alleviates cancer or alleviates a disease condition associated with the PPARγ pathway and HER-kinase axis by at least about 10, 50, 75, 90, or 100% relative to the absence of the NSAID and HER-kinase axis inhibitor, or any percentage therebetween. Animal studies indicate that the method of combination therapy prolongs the life of a mammal with prostate cancer significantly, as compared to a mammal with prostate cancer that does not receive any treatment. Animal studies also indicate that the method inhibits the growth of tumors, has a significant effect in the prevention of metastasis of prostate cancer, and has a noticeable therapeutic effect on prostate cancer.

The above disclosure generally describes the present invention, and all patents and patent applications cited in this disclosure are expressly incorporated by reference herein. A more complete understanding can be obtained by reference to the following Examples, which are provided for purposes of illustration only and are not intended to limit the scope of the invention.

### EXAMPLES

The following examples are typical of the procedures that may be used to treat mammals suffering from cancer or any other PPARγ-sensitive condition, or to evaluate the efficacy of the combination therapy which may be used to treat such mammals in accordance with various embodiments of the present invention. Modifications of these examples will be readily apparent to those skilled in the art who seek to treat mammals whose condition differs from that described herein.

### Example 1

### Preparation of Tumor Models

Four-to six-week-old nude mice were obtained from National Cancer Institute-Charles Rivers Laboratories and maintained in pressurized ventilated caging at the Cedars-Sinai Medical Center vivarium. Male animals were subcutaneously inoculated with minced tumor tissue from androgen-dependent LuCaP-35 xenografts (Buhler, K. R. et al., “LuCaP 35: An Androgen Inducible, Prostate-Specific Antigen Producing Human Prostate Cancer Xenograft,” Proc. Am. Assoc. Cancer Res., Vol. 38 (1997)), and females received the androgen-independent CWRSA6 xenografts, which were obtained by selecting tumors for regrowth and increased serum prostate specific antigen (PSA) after androgen withdrawal (Agus, D. B. et aL, “Prostate Cancer Cell Cycle Regulators: Response to Androgen Withdrawl and Development of Androgen Independence,” J. Natl. Cancer Inst., Vol. 91, No. 21, p. 1869-1876 (1999)). CWRSA6 and LuCaP-35 were chosen since these models have been well characterized in terms of PSA progression, androgen receptor status and dependence on HER-kinase axis. These models represent two distinct stages of prostate cancer i.e., the androgen-dependent (LuCaP-35) stage and the androgen independent (CWRSA6) stage (Corey, E. et al., “LuCaP 35: a new model of prostate cancer progression to androgen independence,” Prostate, Vol. 55, p. 239-246 (2003); Nagabhushan, M. et al., “CWR22: the first human prostate cancer xenograft with strongly androgen dependent and relapsed strains both in vivo and in soft agar,” Cancer Res., Vol. 56, p. 3042-3046 (1996)). The activity of R-etodolac did not appear to distinguish between the androgen-dependent and independent models in terms of its efficacy. Androgens play a critical role in prostate cancer survival and progression which is why androgen ablation therapy is the accepted first line of treatment for metastatic disease. Prostate cancer subsequently progresses to an androgen independent state, the underlying mechanisms for which are not clear. Subsequent studies were focused on the androgen-independent model since there is an urgent need in the field to identify new therapies to treat this cancer state.

All lines were injected together with reconstituted basement membrane (Matrigel obtained from Collaborative Research; Bedford, Mass.), and RPMI 1640 supplemented with 100 μ/ml penicillin and 100 μg/ml streptomycin as described in Nagabhushan, M. et aL, “CWR22: The First Human Prostate Cancer Xenograft with Strongly Androgen-Dependent and Relapsed Strains Both In vivo and in Soft Agar,” Cancer Res., Vol. 56, p. 1058-1061 (1996); Wainstein, M. A. et al, “CWR22: Androgen-Dependent Xenograft Model Derived from a Primary Human Prostatic Carcinoma,” Cancer Res., Vol. 54, p. 6049-6052 (1994)). To maintain serum testosterone levels (for the LuCap-35 line), mice were subcutaneously implanted with 12.5 mg sustained release testosterone pellets (obtained from Innovative Research of America; Sarasota, Florida) one week before receiving the tumor cell inoculation.

### Example 2

### Administering Combination Therapy

Treatments consisted of daily oral gavage of 200 mg/kg R-etodolac (obtained from Salmedix, Inc.; San Diego, Calif.), in water supplemented with 0.5% methycellulose and 0.5% polysorbate 80, for single agent efficacy studies. The combination regimen study consisted of daily oral gavage of 200 mg/kg R-etodolac, and twice weekly intraperitoneal injection of 20 mg/kg 2C4 (obtained from Genentech; San Francisco, Calif.) in phosphate buffered saline (PBS).

### Example 3

### Statistical Analysis of Combination Therapy Efficacy

Tumors were measured every 3-4 days with vernier calipers, and tumor volumes were calculated by the formula: π/6×(larger diameter)×(smaller diameter)². Animals with palpably established tumors of at least 65 mm³ were designated to treatment groups. A time course study was implemented using R-etodolac treated CWRSA6 xenografts. Fourteen-day-old CWRSA6 xenografts with palpably established tumors of at least 1000 mm³ were randomized into 2 cohorts: experimental and control. The experimental groups received daily oral gavage of 200 mg/kg R-etodolac, and were sacrificed at 24 hrs (n=3), 48 hrs (n=3), and 72 hrs (n=3) post initiation of treatment. The control cohort was sacrificed at the start of the study.

Differences between the tumor volumes of the treatment groups were compared over time using a permutation test. The null hypothesis for this test is that treatment has no differential effect on the tumor volumes over time. The statistic (SS_Dev) used to test the hypothesis was the sum of the squared differences between the mean tumor volumes summed over all time points. The statistic reflects the amount by which the trajectories of average tumor volume of the two treatment groups are different (Agus, D. B. et al., “Response of Prostate Cancer to Anti-HER-2/neu Antibody in Androgen-Dependent and -Independent Human Xenograft Models,” Cancer Res., Vol. 59, p. 4761-4764 (1999)).

### Example 4

### RNA Extraction

Total RNA was extracted from prostate tumors or the LNCaP cell line using the TRIZOL reagent (obtained from Invitrogen Corp.; San Diego, Calif.). Samples were heated at 95° C. for 3 minutes and snap-cooled before proceeding with DNase I treatment to prevent RNA/DNA hybridization. DNase I (obtained from Ambion; Austin, Tex.) was used to remove any genomic DNA that might interfere with the reaction. Samples were treated with DNase I for 1 hour at 37° C. The RNA yield was quantified spectrometrically.

### Example 5

### Real-Time Quantitative Reverse Transcription and PCR Analysis

Total RNA was reverse-transcribed (RT) into cDNA and polymerase chain reaction (PCR) was performed in the same reaction using a real-time TAQman One-Step RT-PCR Master Mix Reagents Kit (obtained from Applied Biosystems; Foster City, Calif.). The sequences of the primer/probe sets used for this analysis are as follows. F and R are the forward and reverse primers, respectively, and P is the fluorescent-labeled probe.

TABLE 1

| | | |
|---|---|---|
| Forward Primer | Reverse Primer | Fluorescent-Labeled Probe |

| | | | |
|---|---|---|---|
| Primer set recognizing | SEQ ID NO:1 | SEQ ID NO:2 | SEQ ID NO:3 |
| PPARy1 and PPARy2 | (5′-CGGGCCCT | (5′-CGCCCTCG | (5′TTTGTATGACTCATACAT |
| | GGCAAAAC-3′) | CCTTTGCT-3′) | AAAGTCCTTCCCGCTG-3′) |
| Primer set recognizing | SEQ ID NO:4 | SEQ ID NO:5 | SEQ ID NO:6 |
| PPARy2 | (5′-CCCAGAAAGC | (5′-AATGGCATCTC | (5′-TGATACACTGTCTGCAAAC |
| | GATTCCTTCA- 3′) | TGTGTCAACCA-3′) | ATATCACAAGAAATGACC-3′) |
| Primer set recognizing | SEQ ID NO:7 | SEQ ID NO:8 | SEQ ID NO:9 |
| β-actin | (5′-GCGCGGCT | (5′TCTCCTTAATG | (5′-CACCACGGC |
| | ACAGCTTCA-3′) | TCACGCACGAT-3′) | CGAGCGGGA-3′) |

The mRNA expression of cyclin D1 (Takayasu, H. et al., “Frequent deletions and mutations of the beta-catenin gene are associated with overexpression of cyclin D1 and fibronectin and poorly differentiated histology in childhood hepatoblastoma,” Clin. Cancer Res., Vol. 7, p. 901-908 (2001)) and the HER-kinase receptors were analyzed using primer sets as described in Agus, D. B. et al, “Targeting ligand activated ErbB2 signaling inhibits breast and prostate tumor growth,” Cancer Cell, Vol. 2, p. 127-137 (2002).

The real-time one step RT-PCR cycling conditions for all primer sets were as follows: 30 min at 48° C. for RT step; 10 min at 95° C. for AMPLITAQ Gold Activation; and 40 cycles for cDNA denaturing (95° C., 15 s), and annealing/elongation (60° C. for 1 min) steps. PCR reactions for each template were done in triplicate using 1 μg of total RNA per sample. Each gene-specific primer pair was tested on standard 384-well plates. Standard curves were constructed using 10-1000 ng of total RNA prepared from the CWRSA6 tumor line. All experiments were optimized such that the threshold cycle (CT) from triplicate reactions did not differ by more than one cycle number.

The comparative CT method (Perkin Elmer Applied Biosystems; Foster City, Calif.) was used to determine relative quantification of gene expression for each gene compared with the β-actin control. First, the C_{T} values from the β-actin reactions were averaged for each triplicate. Next, the C_{T} values from the gene-of-interest reactions were averaged. The gene-of-interest average was divided by the β-actin average to take into account the variability of total RNA.

### Example 6

### Western Blot Analysis

Immunoblots were performed as described in Towbin H., “Electrophoretic Transfer of Proteins from Polyacrylamide Gels to Nitrocellulose Sheets: Procedure and Some Applications,” Proc. Natl. Acad. Sci. USA, Vol. 76, No. 9 p. 4350-4354 (1979) using aliquots of tumor lysates (100 μg per lane) or lysates from LNCaP cells (100 μg per lane). Protein concentration was determined with a Bradford Assay (obtained from Bio-Rad Inc.; Hercules, Calif.). The lysates were separated on 10% SDS polyacrylamide gels and transferred onto nitrocellulose filters. The membranes were incubated overnight at 4° C. with antibodies against PPARγ (Sc-7273 obtained from Santa Cruz Inc.; Santa Cruz, Calif.), ERK1 (Sc-94 obtained from Santa Cruz Inc.; Santa Cruz, Calif.), and phospho-p44/42 MAP kinase (9101S obtained from Cell Signal; Beverly, Mass.), and cyclin D1 (554180 obtained from BD Pharmingen; San Diego, Calif.) in parallel with anti-β-actin antibodies (A2066 obtained from Sigma; St. Louis, Mo.) and washed well before incubating with the appropriate α-mouse or α-rabbit secondary antibody conjugated with horseradish peroxidase (obtained from Amersham Biosciences, UK).

### Example 7

### Maintenance of Cell Cultures

Raw 267.4 cells were maintained at 37° C. and 5% CO₂ in Dulbecco's modified eagle medium (DMEM) with high glucose (obtained from GIBCO; Grand Island, N.Y.) supplemented with 10% fetal bovine serum (FBS), 100 μg/ml penicillin and 100 μg/ml streptomycin.

### Example 8

### Transfection

For transfection, cells were grown in DMEM with 10% FBS for at least 24 hours prior to transfection. Transient transfections were performed in 12-well plates for at least 24 hours prior to transfection. At approximately 50% confluence, cells were transfected using the FuGENE transfection reagent (obtained from Roche Diagnostics GmbH; Mannheim, Germany) according to the manufacturer's instruction with 0.5 μg of reporter plasmid (AOx)₃-TK-Luc, 0.1 to 0.2 μg of control plasmid pCMXμgal, 0.1 μg PPARγ expression plasmid and carrier DNA for a total of 1 μg DNA per well. After 16 hours, the cells were washed and fresh medium containing the appropriate amount of drugs, prepared in 0.5% DMSO, was added to the cells for another 24 hours. The cells were treated with either vehicle alone (DMSO), indomethacin, rosiglitazone or varying concentrations of R- or S-etodolac.

For luciferase assays, cells were lysed in potassium phosphate buffer containing 1% TRITON X-100 (obtained from Rohm and Haas Co.; Philadelphia, Pa.) and light emission was detected in the presence of luciferin using a microtiter plate luminometer. Luciferase values were normalized for variations in transfection efficiency using a β-galactosidase internal control. The results are expressed as relative luciferase units (RLU). The luciferase activity values represent the mean of a minimum of three independent transfections performed in triplicate.

### Example 9

### R-etodolac Transactivates PPARγ

Prostate carcinoma cell lines, 22Rv1 and LNCaP, and human acute monocytic leukemia (THP-1) cells were obtained from the American Type Culture Collection (Rockville, Md.) and maintained in RPMI 1640 medium supplemented with 10% FBS, 100 μg/ml penicillin and 100 μg/ml streptomycin (obtained from GIBCO; Grand Island, N.Y.). Three different assays demonstrate that R-etodolac has the ability to function as a PPARγ transactivator.

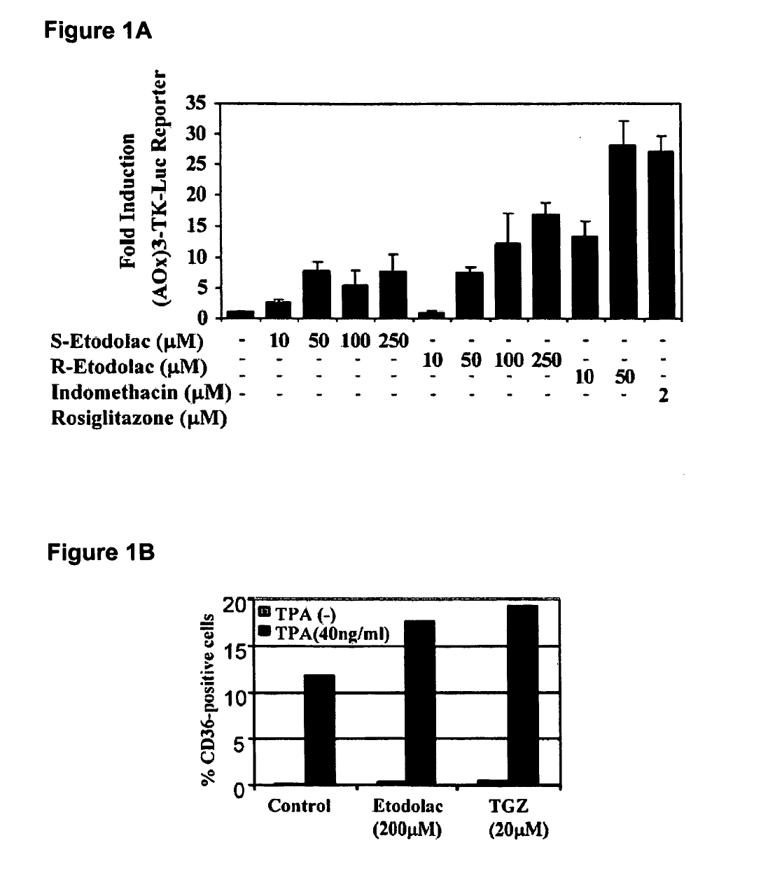

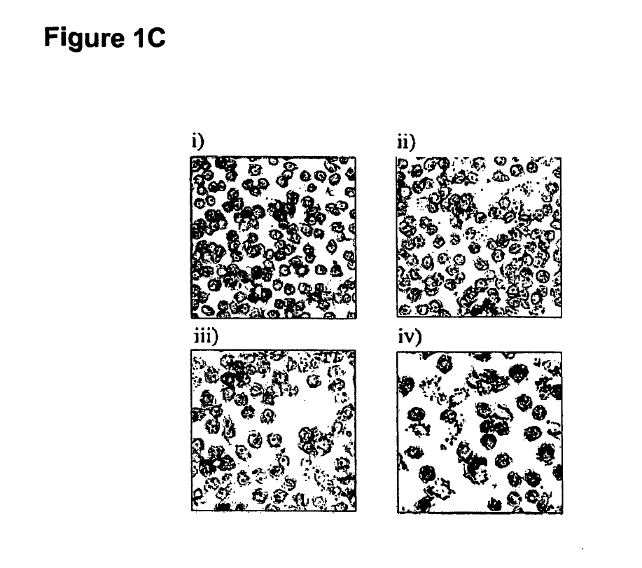

First, R-etodolac enhances reporter gene expression in a dose dependent manner driven by promoters containing PPRE sequences in transient transfection assays (FIG. 1A). The 22Rv1 cells were seeded into 100 mm dishes and allowed to attach for a period of 18 hours. The media was then replaced with phenol red-free and serum-free RPMI 1640, with 100 μg/ml penicillin and 100 μg/ml streptomycin ±R-etodolac for the indicated times. MAP-kinase activation was assessed by Western blot analysis using methods described above. LNCaP cells were treated with R-etodolac at 200, 400, and 600 μM concentrations for 18 Hours. Total protein or RNA was isolated using methods as described previously. Cyclin D1 protein and mRNA expression were assessed by western blot and a real-time quantitative RT-PCR assay as previously described. This transactivation function is mediated only in the presence of recombinant PPARγ. The COX inhibitory analog of etodolac, S-etodolac, can also transactivate PPARγ but to significantly lower levels. Transactivation by 100 μM R-etodolac (13-fold) is comparable to 10 μM of the known PPARγ ligand and NSAID, indomethacin, suggesting that R-etodolac has PPARγ transactivation activity.

Rosiglitazone, another demonstrated PPARγ ligand, was used as a positive control. The two other assays support the notion that R-etodolac is a PPARγ transactivator by showing that CD36 expression and the uptake of neutral lipids as a marker of adipocyte differentiation in the presence of R-etodolac and PPARγ.

Second, the combination of PPARγ transactivation, together with protein kinase C activation by a phorbol ester, has been reported to induce the scavenger receptor CD36 in macrophages (Han and Sidell, 2002). CD36 is documented as a PPARγ-regulated gene (Tontonoz et al., 1998). Using CD36 expression as a downstream indicator, 200 μM of R-etodolac and 20 μM of troglitazone (a demonstrated PPAR-γ activator) displayed equivalent potency as measured by a flow cytometric assay (FIG. 1B). THP-1 cells were treated for 5 days with 200 μM etodolac (racemic mixture) and 20 μM troglitazone in the presence or absence of 40 ng/ml phorbol ester (TPA) for 48 hours. Cells were fixed and stained with unconjugated anti-CD36 and FITC conjugated anti-IgG and analyzed by flow cytometry. A population of 10,000 viable cells was analyzed for each treatment.

Third, PPARγ is accepted as a master regulator of adipocyte differentiation. Uptake of neutral lipids is a marker of adipocyte differentiation and Oil Red O staining of these neutral lipids is an accepted procedure to demonstrate this differentiation phenomenon (Tontonoz et al., 1998). NIH3T3 cells that stably overexpress retrovirally expressed recombinant PPARγ were obtained from Dr. Ronald Evans (Salk Institute, La Jolla, Calif.). Cells treated with 1 μM R-etodolac displayed accumulation of neutral lipids and morphological changes associated with PPARγ activity that are comparable to those observed with troglitazone at a similar concentration (FIG. 1C). NIH3T3 cells stably expressing recombinant PPARγ (obtained from Dr. Ronald Evans of the Jonas Salk Institute; San Diego, Calif.) were maintained in DMEM supplemented with 10% BCS, 100 μg/ml penicillin and 100 μg/ml streptomycin (obtained from GIBCO; Grand Island, N.Y.). The cells were treated for seven days with the indicated compounds and concentrations and stained for neutral lipids with Oil Red O as described by Green and Kehinde (“Sublines of mouse 3T3 cells that accumulate lipid,” Cell, Vol. 1, p. 113-116 (1974)). The lipid uptake was dose-dependent and was significantly more pronounced at 500 μM concentration of R-etodolac. NIH3T3 cells transfected with the empty retroviral vector did not demonstrate the lipid uptake with either troglitazone or R-etodolac (data not shown). Having demonstrated that PPARγ could be positively modulated by R-etodolac, the possibility of using it as a potential therapeutic against prostate tumor models was considered.

### Example 10

### Growth Inhibition of Prostate Cancer Xenografts by R-etodolac

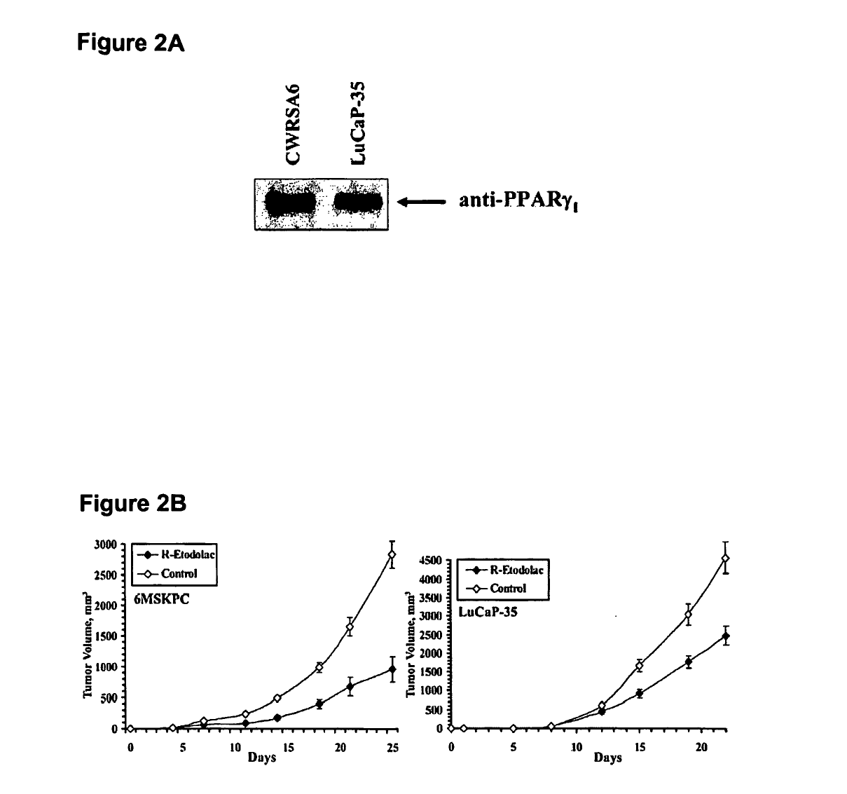

Immunoblot analyses demonstrated the presence of PPARγ₁ in the androgen-dependent LuCaP-35 and androgen-independent CWRSA6 xenografts (FIG. 2A). Real-time RT-PCR further confirmed the presence of PPARγ mRNA in the xenografts (data not shown). Primers specific for PPARγ₂ did not yield an appreciable amplification whereas total PPARγ-specific primers led to a robust amplification suggesting that the predominant isoform in the prostate cancer xenografts was PPARγ₁. To examine the effect of R-etodolac treatment on tumor cell growth, animals with established androgen-independent CWRSA6 or androgen-dependent LuCaP-35 prostate cancer xenografts were administered R-etodolac (200 mg/kg o.g. daily). At this dose no lethal toxicity or weight loss (greater than 10% body weight) was observed amongst treated animals and a drug concentration of 500 μM was achieved in the serum (data not shown). R-etodolac demonstrated significant tumor growth inhibition in the androgen independent CWRSA6 xenograft (66% growth inhibition, p=0.002, n=10) and androgen dependent LuCaP-35 xenografts (46% growth inhibition, p=0.0103, n=10) relative to control-treated animals (FIG. 2B). These studies demonstrate that R-etodolac inhibits the growth of PPARγ-positive prostate cancer xenografts.

### Example 11

### R-etodolac Causes a Downregulation in Cyclin D1 Expression

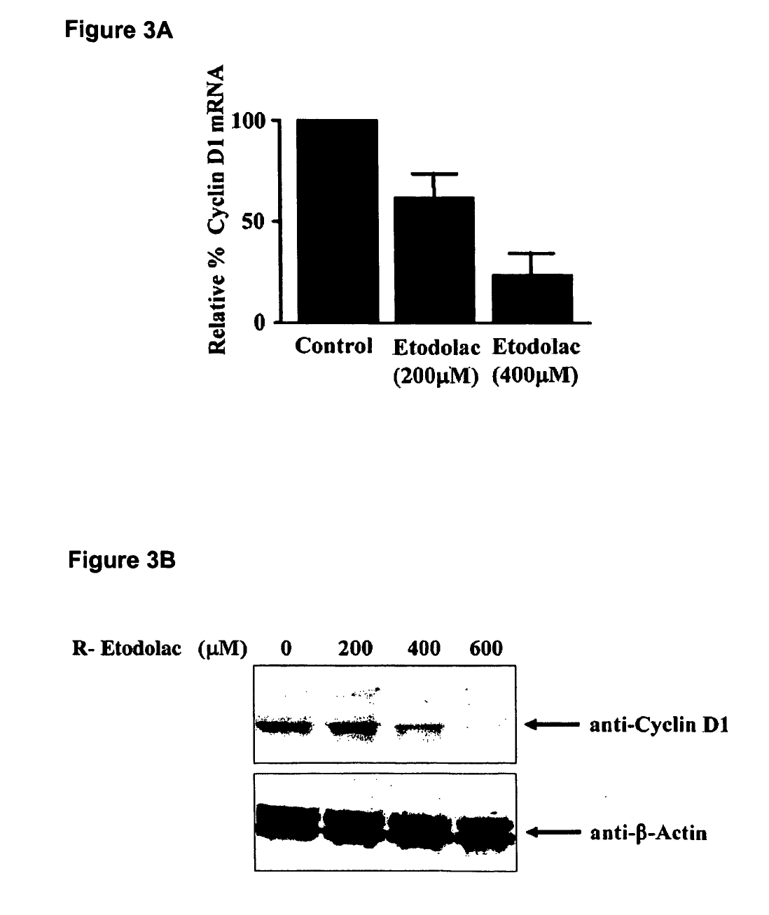

PPARγ transactivation is well documented to have an effect on cell cycle progression through the repression of the cyclin D1 promoter (Qin, C. et al., “Peroxisome proliferator-activated receptor gamma agonists induce proteasome-dependent degradation of cyclin D1 and estrogen receptor alpha in MCF-7 breast cancer cells,” Cancer Res., Vol. 63, p. 958-964 (2003); Wang, C. et al., “Cyclin D1 repression of peroxisome proliferator-activated receptor gamma expression and transactivation,” Mol. Cell Biol, Vol. 23, p. 6159-6173 (2003)). Accordingly, we observed a downregulation in cyclin D1 mRNA (FIG. 3A) and protein (FIG. 3B) expression following 18 hours of R-etodolac treatment of LNCaP prostate cancer cells. The decrease in cyclin D1 expression was dependent upon increasing concentrations of R-etodolac. The inhibition of cyclin D1 expression by R-etodolac provides insight into the mechanism by which R-etodolac inhibits tumor growth.

### Example 12

### R-etodolac Treatment Leads to a Degradation of PPARγ Protein

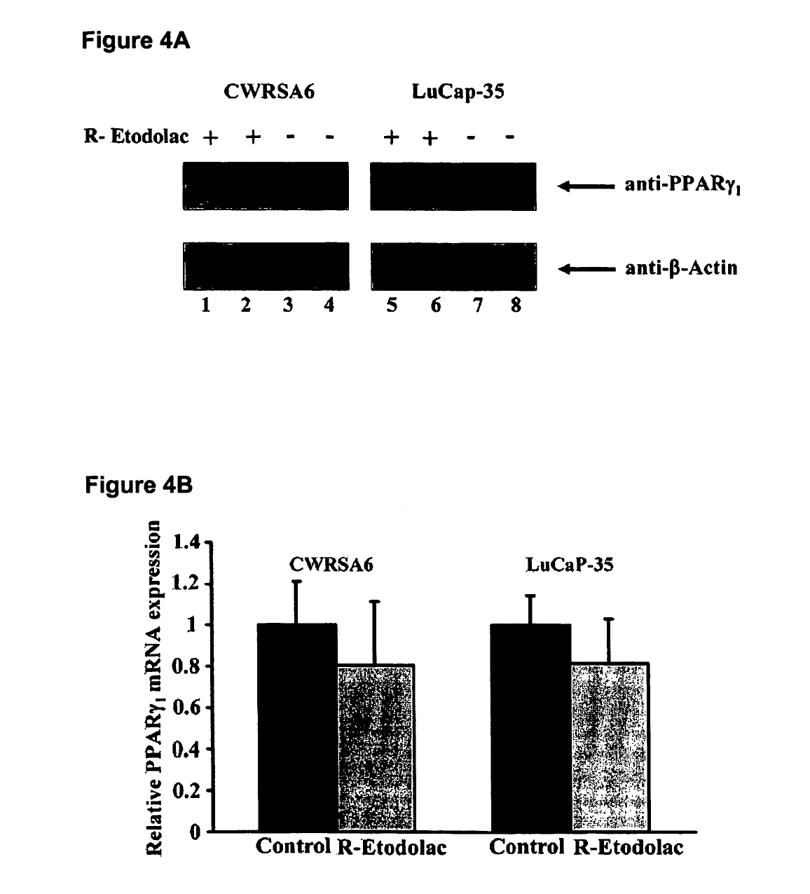

PPARγ protein levels were analyzed in prostate xenografts following R-etodolac treatment to elucidate the growth inhibitory mechanism of R-etodolac on prostate xenografts. Since transient transfection studies indicated that R-etodolac serves as a PPARγ transactivator, an upregulation was expected or no change in PPARγ protein since the drug had caused a significant reduction in tumor growth. Surprisingly, the PPARγ protein was completely eliminated from the tumors following two to three weeks of drug treatment as compared to the vehicle treated control (FIG. 4A). Real time RT-PCR analysis of PPARγ₁ mRNA at this time point showed that it was similar to that in the control animals (FIG. 4B), indicating that PPARγ downregulation was predominantly a post-transcriptional event.

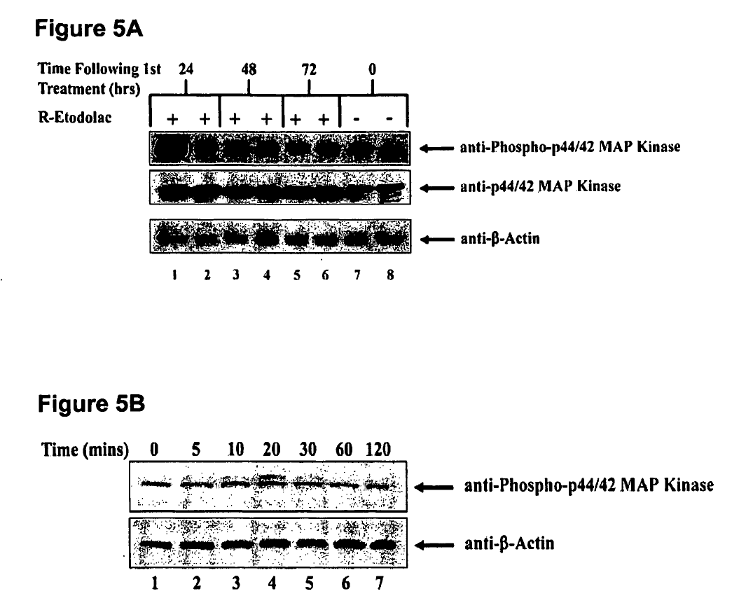

Phospho-ERK1 and phospho-ERK2 levels were analyzed following R-etodolac treatment to elucidate whether PPARγ phosphorylation by MAP kinase led to negative regulation of the nuclear receptor's function via the ubiquitin-proteosome-mediated-degradation pathway (Floyd, Z. E., and J. M. Stephens et al., “Interferon-gamma-mediated activation and ubiquitin-proteasome-dependent degradation of PPARgamma in adipocytes,” J. Biol. Chem., Vol. 277, p. 4062-4068 (2002)). Using a phospho-specific MAP kinase antibody, a sharp increase in phosphorylated p44/42 levels was observed 24 hours post-R-etodolac treatment (FIG. 5A). This transient increase was followed by a low level of sustained activation of MAP kinase throughout the rest of the time course up to 72 hours. At 3 weeks post drug treatment, immunoblot analysis of phospho-ERK1/2 protein appeared similar to that at 72 hrs (data not shown). No difference was observed in total MAP kinase expression and equal loading was confirmed with an anti-β-actin antibody. These results in the prostate cancer xenograft were later confirmed in vitro by treating 22Rv1 prostate cancer cells with a concentration of R-etodolac (500 μM) physiologically similar to that achieved in the xenograft studies (data not shown). A transient peak in phospho-MAP kinase activation, although modest as compared to the in vivo studies, was observed within 20 minutes of the addition of R-etodolac and then maintained a sustained level of activation (FIG. 5B). Such transient upregulation in phospho-MAP kinase activation also observed with troglitazone treatment ultimately led to degradation of PPARγ protein (Baek, S. J. et al., “Troglitazone, a Peroxisome Proliferator-Activated Receptor γ (PPARγ) Ligand, Selectively Induces the Early Growth Response-1 Gene Independently of PPARγ. A Novel Mechanism for it Anti-Tumorigenic Activity,” J. Biol. Chem., Vol. 278, No. 8, p. 5845-5853 (2003)). These results suggest that R-etodolac (200 mg/kg o.g. daily) treatment exerts two opposing effects on tumor growth. Not only does R-etodolac increase the anti-tumorigenic potential of PPARγ as demonstrated in the transient transfection studies, but it may also regulate the levels of PPARγ protein via upregulation of phospho-ERK1/2, which may have a consequence on the anti-tumorigenicity of R-etodolac.

### Example 13

### Suppression of Prostate Tumor Growth via Combination Therapy

Since treatment with R-etodolac resulted in an up-regulation of phospho-MAP kinase, and subsequent degradation of PPARγ protein, inhibiting phospho-MAP kinase should increase the efficacy of R-etodolac. To prevent MAP kinase activation and subsequent PPARγ degradation, CWRSA6 xenografts were treated with a regimen of R-etodolac in combination with 2C4. This inhibitor, 2C4, is a humanized monoclonal antibody that abrogates MAP kinase activation by sterically inhibiting ligand-induced heterodimerization of ErbB2 with members of the HER-kinase receptor family (Agus, D. B. et al., (2002)).

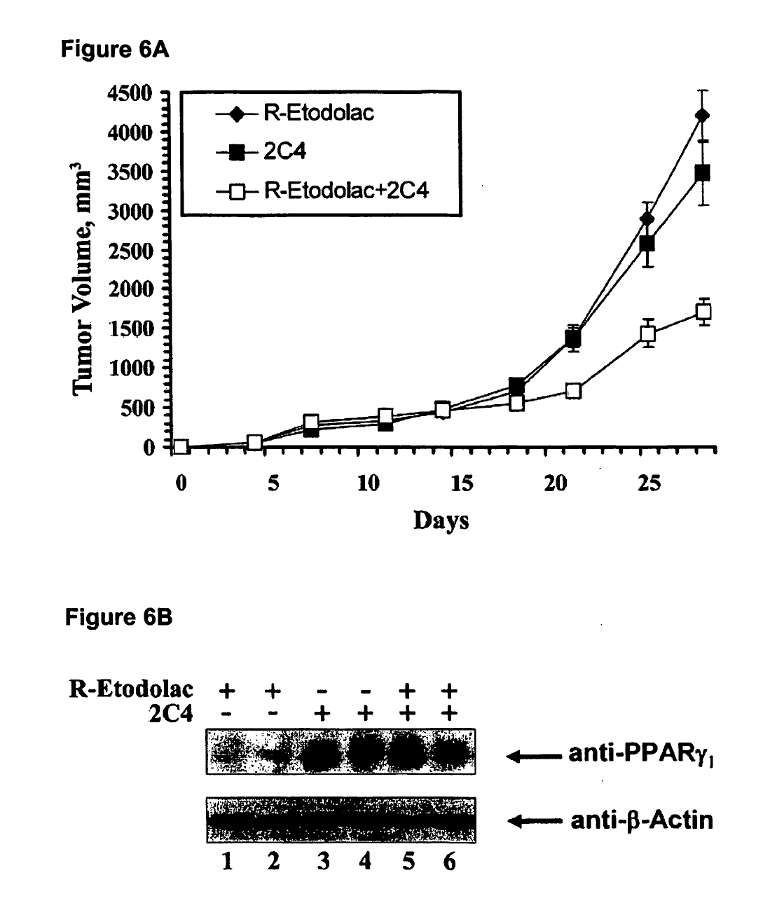

Mice bearing established CWRSA6 xenografts received monotherapy with either R-etodolac (200 mg/kg o.g. daily n=10) or 2C4 (20 mg/kg I.P. 2X/wk n=10), or a combination regimen of R-etodolac and 2C4 for three weeks (n=10). At these doses no lethal toxicity or significant weight loss was observed among treated animals. CWRSA6 xenografts receiving a combination regimen of R-etodolac and 2C4 demonstrated significant tumor inhibition as compared to animals receiving R-etodolac monotherapy (tumor inhibition 59%, p<0.001, n=10), and 2C4 alone (tumor inhibition 51%, p=0.001, n=10) (FIG. 6A). These results support the hypothesis that the efficacy of R-etodolac can be improved by inhibiting phospho-MAP kinase.

One prediction in accordance with this hypothesis would be the rescue of PPARγ protein from degradation following the combination treatment. To ascertain this, total cell lysates were prepared from xenografts in the preceding study, and were subjected to immunoblot analysis for PPARγ protein. As expected, samples treated with R-etodolac alone had a complete ablation of the PPARγ protein (FIG. 6B, lanes 1 and 2), and animals receiving a combination regimen of R-etodolac and 2C4 maintained their PPARγ protein expression (FIG. 6B, lanes 5 and 6). Xenografts treated with 2C4 alone also expressed PPARγ (FIG. 6B, lanes 3 and 4). R-etodolac treatment did not alter the mRNA expression of the HER-kinase receptors (data not shown).

While the description above refers to particular embodiments of the present invention, it will be understood that many modifications may be made without departing from the spirit thereof. For instance, the composition or a similar composition of the present invention may be used in the treatment of any number of disease conditions where observed, as would be readily recognized by one skilled in the art and without undue experimentation. The accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

What is claimed is:

1. A composition for treating a condition in a mammal, comprising:
a non-steroidal anti-inflammatory drug (NSAID); and
a HER-kinase axis inhibitor.

2. The composition of claim 1, wherein the NSAID is selected from the group consisting of aspirin, diclofenac, diflunisal, etodolac, fenoprofen, floctafenine, flurbiprofen, ibuprofen, indomethacin, ketoprofen, meclofenamate, mefenamic acid, meloxicam, nabumetone, naproxen, oxaprozin, piroxicam, sunlindac, tenoxicam, tiaprofenic acid, tolmetin, derivatives thereof, analogs thereof, pharmaceutical equivalents thereof, and combinations thereof.

3. The composition of claim 1, wherein the HER-kinase axis inhibitor is selected from the group consisting of recombinant humanized monoclonal antibody 2C4, ansamycins, gefitinib, erlotinib, monoclonal antibodies, rapamycin, src inhibitors, tyrosine kinase inhibitors, compound LY294002, imatinib mesylate, trastuzumab, compound CI1033, compound PKI166, compound GW2016, compound EKB569, compound IMC-C225, and derivatives thereof, analogs thereof, pharmaceutical equivalents thereof, and combinations thereof.

4. The composition of claim 1, wherein said NSAID is R-etodolac.

5. The composition of claim 1, wherein said HER-kinase axis inhibitor is recombinant humanized monoclonal antibody 2C4.

6. The composition of claim 1, comprising a quantity of said NSAID of from about 100 to about 500 mg/kg of said mammal.

7. The composition of claim 1, comprising a quantity of said HER-kinase axis inhibitor of from about 5 to about 40 mg/kg of said mammal.

8. The composition of claim 1, wherein the composition is formulated for administration by a technique selected from the group consisting of intraperitoneal, oral gavage, intravenous, sublingual, topical, intramuscular, intra-arterial, intramedullar, intrathecal, intraventricular, transdermal, subcutaneous, intranasal, parenteral, and rectal.

9. The composition of claim 1, further comprising an additional component selected from the group consisting of a vehicle, an additive, an excipient, a therapeutic compound, a pharmaceutical adjunct, a pharmaceutical agent useful in the treatment of the condition, a carrier and combinations thereof.

10. A method of treating a condition in a mammal, comprising:
administering a quantity of a non-steroidal anti-inflammatory drug
(NSAID) to said mammal on a NSAID periodic basis; and
administering a quantity of a HER-kinase axis inhibitor to said mammal on a HER-kinase axis inhibitor periodic basis.

11. The method of claim 10, wherein the NSAID is selected from the group consisting of aspirin, diclofenac, diflunisal, etodolac, fenoprofen, floctafenine, flurbiprofen, ibuprofen, indomethacin, ketoprofen, meclofenamate, mefenamic acid, meloxicam, nabumetone, naproxen, oxaprozin, piroxicam, sunlindac, tenoxicam, tiaprofenic acid, tolmetin, derivatives thereof, analogs thereof, pharmaceutical equivalents thereof, and combinations thereof.

12. The method of claim 10, wherein the HER-kinase axis inhibitor is selected from the group consisting of recombinant humanized monoclonal antibody 2C4, ansamycins, gefitinib, erlotinib, monoclonal antibodies, rapamycin, src inhibitors, tyrosine kinase inhibitors, compound LY294002, imatinib mesylate, trastuzumab, compound CI1033, compound PKI166, compound GW2016, compound EKB569, compound IMC-C225, and derivatives thereof, analogs thereof, pharmaceutical equivalents thereof, and combinations thereof.

13. The method of claim 10, wherein said NSAID is R-etodolac.

14. The method of claim 10, wherein said HER-kinase axis inhibitor is recombinant humanized monoclonal antibody 2C4.

15. The method of claim 10, wherein the quantity of said NSAID is from about 100 to about 500 mg/kg of said mammal.

16. The method of claim 10, wherein the quantity of said HER-kinase axis inhibitor is from about 5 to about 40 mg/kg of said mammal.

17. The method of claim 10, wherein the HER-kinase axis inhibitor periodic basis is twice weekly.

18. The method of claim 10, wherein the NSAID periodic basis is daily.

19. The method of claim 10, wherein administering said quantity of said NSAID and administering said quantity of said HER-kinase axis inhibitor further comprises using a delivery technique independently selected from the group consisting of intraperitoneal, oral gavage, intravenous, sublingual, topical, intramuscular, intra-arterial, intramedullar, intrathecal, intraventricular, transdermal, subcutaneous, intranasal, parenteral, and rectal.

20. A kit for use in treating a condition in a mammal, comprising:
a quantity of a non-steroidal anti-inflammatory drug (NSAID);
a quantity of a HER-kinase axis inhibitor; and
instructions for use of said NSAID and said HER-kinase axis inhibitor in the treatment of said condition.

21. The kit of claim 20, wherein the NSAID is selected from the group consisting of aspirin, diclofenac, diflunisal, etodolac, fenoprofen, floctafenine, flurbiprofen, ibuprofen, indomethacin, ketoprofen, meclofenamate, mefenamic acid, meloxicam, nabumetone, naproxen, oxaprozin, piroxicam, sunlindac, tenoxicam, tiaprofenic acid, tolmetin, derivatives thereof, analogs thereof, pharmaceutical equivalents thereof, and combinations thereof.

22. The kit of claim 20, wherein the HER-kinase axis inhibitor is selected from the group consisting of recombinant humanized monoclonal antibody 2C4 (2C4), ansamycins, gefitinib, erlotinib, monoclonal antibodies, rapamycin, src inhibitors, tyrosine kinase inhibitors, compound LY294002, imatinib mesylate, trastuzumab, compound CI1033, compound PKI166, compound GW2016, compound EKB569, compound IMC-C225, and derivatives thereof, analogs thereof, pharmaceutical equivalents thereof, and combinations thereof.

23. The kit of claim 20, wherein said NSAID is R-etodolac.

24. The kit of claim 20, wherein said HER-kinase axis inhibitor is recombinant humanized monoclonal antibody 2C4.

25. The kit of claim 20, wherein the instructions for use indicate that at least a portion of said quantity of said NSAID is to be administered to said mammal on an NSAID periodic basis, and that at least a portion of said quantity of said HER-kinase axis inhibitor is to be administered on a HER-kinase axis inhibitor periodic basis.

26. The kit of claim 25, wherein the HER-kinase axis inhibitor periodic basis is twice weekly.

27. The kit of claim 25, wherein the NSAID periodic basis is daily.

28. The kit of claim 20, wherein said NSAID and said HER-kinase axis inhibitor are each formulated for administration by a delivery technique independently selected from the group consisting of intraperitoneal, oral gavage, intravenous, sublingual, topical, intramuscular, intra-arterial, intramedullar, intrathecal, intraventricular, transdermal, subcutaneous, intranasal, parenteral, and rectal.
